(19) United States
(12) Patent Application Publication
(10) Pub. No.: US 2026/0140391 A1
(43) Pub. Date: May 21, 2026

(54) Multifunctional Eyewear Assembly Device

(71) Applicant: Miguel Angel Ramirez, San Francisco, CA (US)

(72) Inventor: Miguel Angel Ramirez, San Francisco, CA (US)

(21) Appl. No.: 19/275,432

(22) Filed: Jul. 21, 2025

Related U.S. Application Data
(63) Continuation-in-part of application No. 29/991,304, filed on Feb. 27, 2025.
(60) Provisional application No. 63/721,834, filed on Nov. 18, 2024.

(51) Int. Cl. G02C 5/00; G02C 5/22; G02C 9/02; G02C 11/00; G02C 13/00

(52) U.S. Cl. CPC G02C 5/001; G02C 9/02; G02C 11/12; G02C 13/001; G02C 5/2209

(57) ABSTRACT

A multi-functional safety eyewear device with an inbuilt reading eyewear is provided. To accomplish this, the device includes a pair of reading glasses that are integrated onto the safety glasses in such a way that the user may use the reading glasses when needed without removing the safety glasses. More specifically, the reading glasses are attached to the sides of the safety glasses in a slidable fashion. Further, the reading glasses may be attached to the safety glasses through multiple means. Furthermore, a pair of grommets attached to the sides of the safety eyewear enables easy attachment and detachment of the reading eyewear. Thus, the flexibility in the ways in which the reading glasses may be mounted onto the safety glasses as well as the adjustable functionality makes the device a user-friendly and efficient eye-wear device.

### FIELD OF THE INVENTION

The present invention relates generally to safety glasses. More specifically, the present invention is an adjustable and multifunctional pair of safety glasses, wherein a pair of reading glasses are integrated into the safety glasses.

### BACKGROUND OF THE INVENTION

Safety glasses provide physical protection to shield the eyes against debris. In addition to physical protection, safety glasses or goggles are the primary protectors intended to shield the eyes against liquid or chemical splashes, irritating mists, vapors, and fumes. They form a protective seal around the eyes and prevent objects or liquids from entering under or around the goggles. Reading glasses, also known as readers or cheater glasses, are used to improve near vision and make it easier to read and complete other close-up tasks. More specifically, reading glasses are designed to temporarily compensate for the effects of presbyopia, which is the natural loss of elasticity in the eyes that occurs with age. They can be purchased over the counter at drug stores and supermarkets. However, while using safety glasses people who need reading glasses for either reading or any close-up tasks will have a hard time removing the safety glasses first and put on the reading glass for reading something quick.

An objective of the present invention is to provide users with a multi-functional eye wear assembly device. To accomplish this, the present invention comprises a pair of safety glasses and a pair of reading glasses that are integrated onto the safety glasses, such that the user may use the reading glasses when needed without removing the safety glasses. According to the present invention, the reading glasses may be attached to the safety glasses through multiple means. Thus, the flexibility in the ways in which the reading glasses may be mounted onto the safety glasses as well as the adjustable usability makes the present invention a user-friendly and efficient eye-wear device.

### SUMMARY OF THE INVENTION

The present invention is an eyewear device intended to provide users with a multi-functional safety glasses device with an inbuilt reading glass. To accomplish this, the present invention comprises a pair of reading glasses that are integrated onto the safety glasses in such a way that the user may use the reading glasses when needed by sliding the reading glasses up and down, without removing the safety glasses. More specifically, the reading glasses are attached to the sides of the safety glasses in a slidable fashion. According to the present invention, the reading glasses may be attached to the safety glasses through multiple means. Further, a pair of grommets attached to the sides of the safety eyewear enables easy attachment and detachment of the reading eyewear to and from the safety eyewear as well. Thus, the flexibility in the ways in which the reading glasses may be mounted onto the safety glasses as well as the adjustable functionality makes the present invention a user-friendly and efficient eye-wear device.

### BRIEF DESCRIPTION OF THE DRAWINGS

FIG. 1 is a top-front-left perspective view of the present invention in a reading configuration, wherein the reading eyewear is positioned in front of the safety eyewear.

FIG. 2 is a bottom-rear-right perspective view of the present invention in the reading configuration.

FIG. 3 is an exploded bottom-rear-right perspective view of the present invention.

FIG. 4 is a front elevational view of the present invention.

FIG. 5 is a rear elevational view of the present invention.

FIG. 6 is a top plan view of the present invention.

FIG. 7 is a left side elevational view of the present invention.

FIG. 8 is top-front-left perspective view of the present invention in a non-reading configuration, wherein the reading eyewear is slid upwards.

FIG. 9 is a side elevational view of the present invention in the non-reading configuration.

FIG. 10 is a front elevational view of the present invention in the non-reading configuration.

### DETAILED DESCRIPTION OF THE INVENTION

All illustrations of the drawings are for the purpose of describing selected versions of the present invention and are not intended to limit the scope of the present invention.

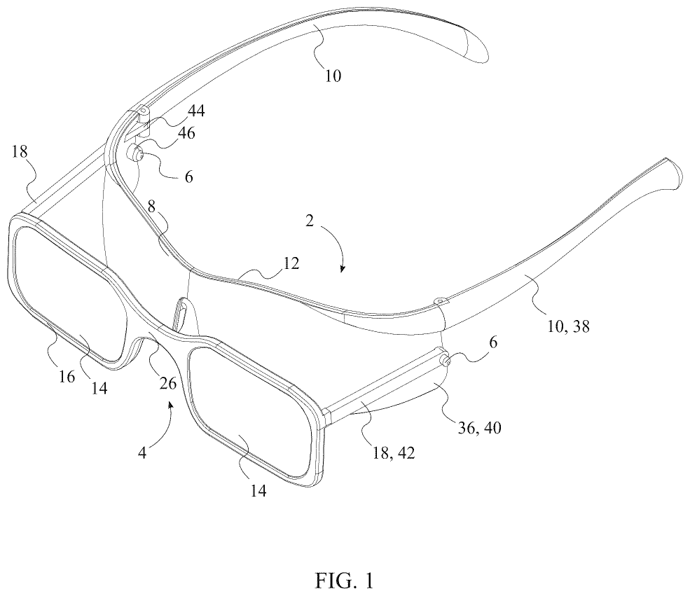

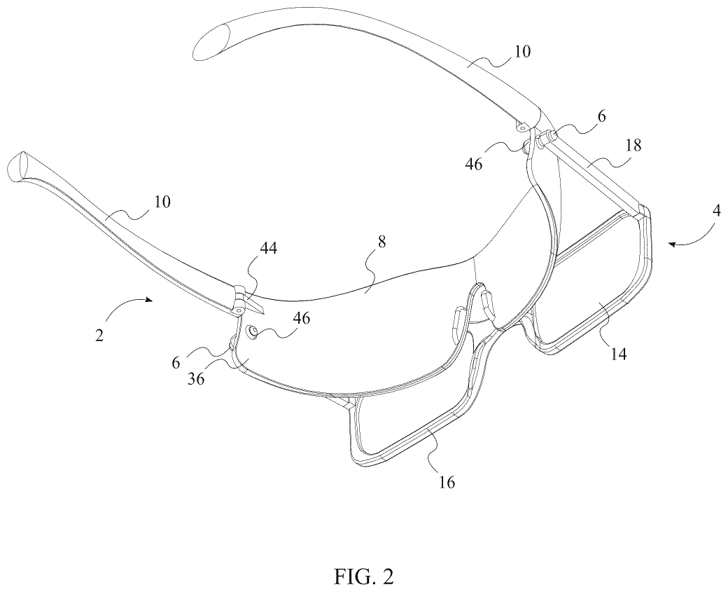

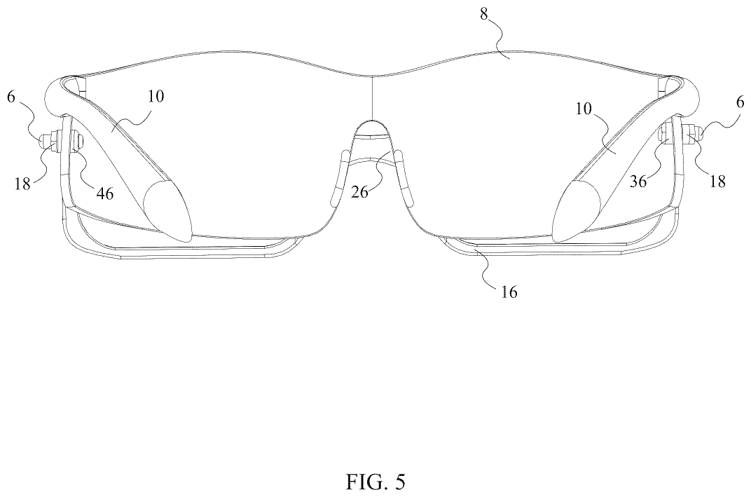

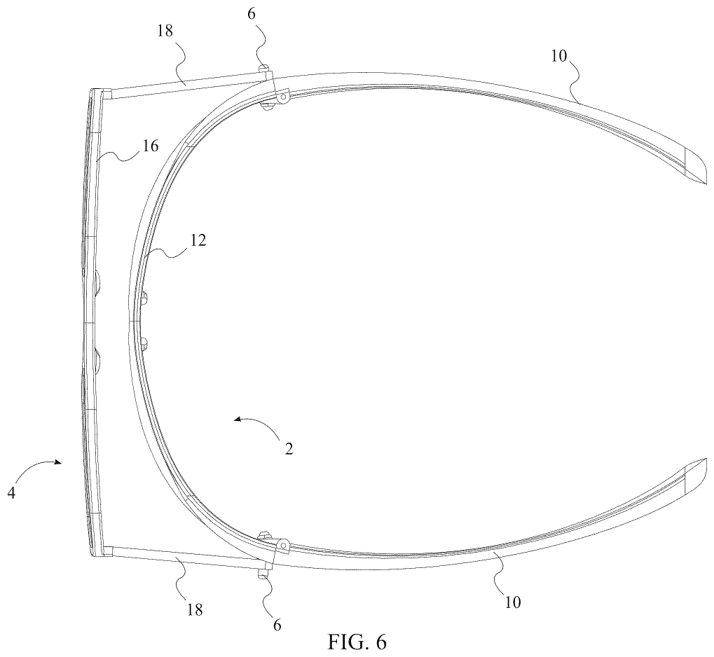

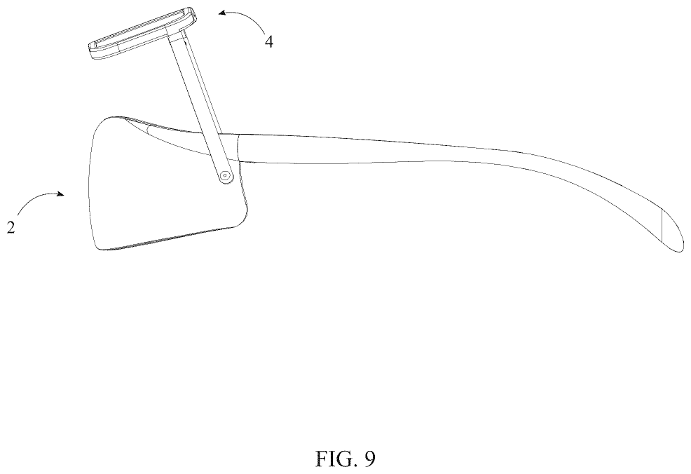

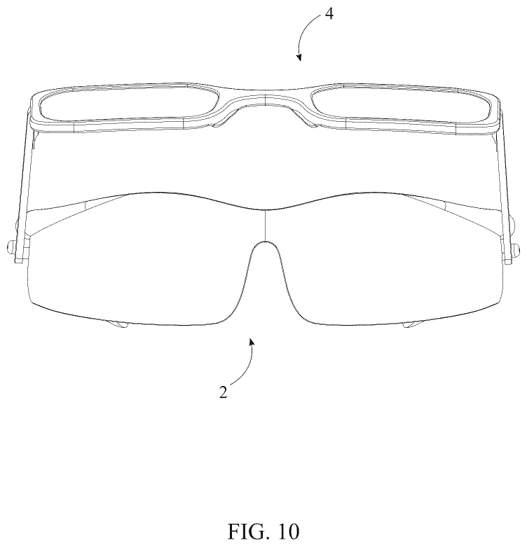

In reference to FIG. 1 through FIG. 10, the present invention is a multifunctional eyewear assembly device.

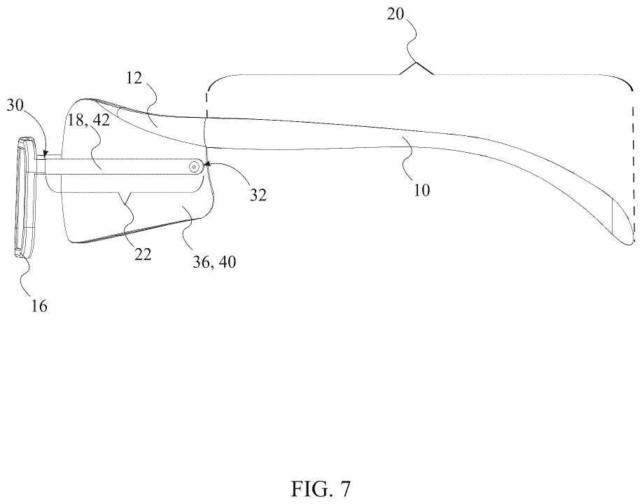

The following description is in reference to FIG. 1 through FIG. 10. According to a preferred embodiment, the present invention comprises a safety eyewear 2, a reading eyewear 4, and a plurality of fasteners 6. In the preferred embodiment, the safety eyewear 2 is transparent with side coverings that protect a user's eyes from all directions. Accordingly, the safety eyewear 2 comprises a pair of safety lenses 8, a pair of temples 10, and a safety frame 12. As seen in FIG. 1 through FIG. 10, each of the pair of temples 10 is terminally mounted onto opposing ends of the safety frame 12. In other words, each of the pair of temples 10 or arms of the safety eyewear 2 that goes behind the ears of the user, is attached to one end of the safety frame 12 on the opposing sides. Further, the pair of safety lenses 8 is also mounted onto the safety frame 12. In an alternate embodiment, the safety eyewear 2 may be a pair of safety goggles. In this embodiment, the pair of safety glasses 8 are transparent with side coverings that protect a user's eyes from all directions and touch the skin around the user's eye area in a snug fashion. It should be noted that the pair of safety glasses 8 may comprise any size, brand, attachment means, material, components, arrangement of components, etc. that are known to one of ordinary skill in the art, as long as the intents of the present invention are not altered. Further, the reading eyewear 4 comprises a pair of reading lenses 14, a reading frame 16, and a pair of shortened arms 18. The reading eyewear 4 comprises normal glasses that solve near vision problems. As seen in FIG. 1 through FIG. 10, the pair of reading lenses 14 is mounted within the reading frame 16, and the pair of shortened arms 18 is mounted on to the reading frame 16. In the preferred embodiment, the legs of the reading eyewear 4 are shortened or less than normal eyewear legs. More specifically, and as seen in FIG. 7, a first length 20 of each of the pair of temples 10 is longer than a second length 22 of each of the pair of the shortened arms 18. This is so that the terminal ends of the pair of shortened arms 18 may be mounted onto the sides of the pair of safety glasses 8. In other words, the lengths of the legs of the reading eyewear 4 are made in such a way that they enable the reading eyewear to be positioned in front of the safety glasses as well as be slid over or glided above the safety eyewear 2 as preferred by the user. To accomplish this, the reading eyewear 4 is slidably engaged onto the safety eyewear 2, and the plurality of fasteners 6 is connected between the pair of safety lenses 8 and the shortened arms 18. Preferably, the positioning of the reading glasses does not affect the functionality of both the reading glasses as well as the safety glasses. Thus, the reading eyewear 4 is operatively engaged to the safety eyewear 2 through the plurality of fasteners 6, wherein the safety eyewear 2 slidably switches between a reading configuration and a non-reading configuration. In other words, the multifunctional safety eyewear may be used in both a reading mode as well as in a stowed configuration or non-reading mode.

A more detailed description of the present invention follows.

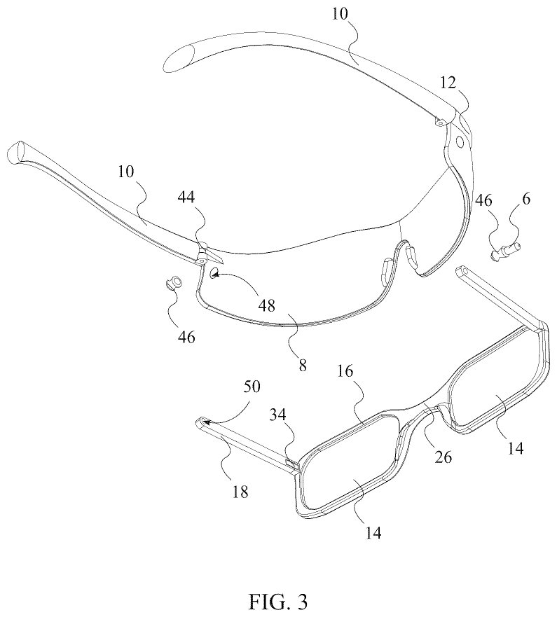

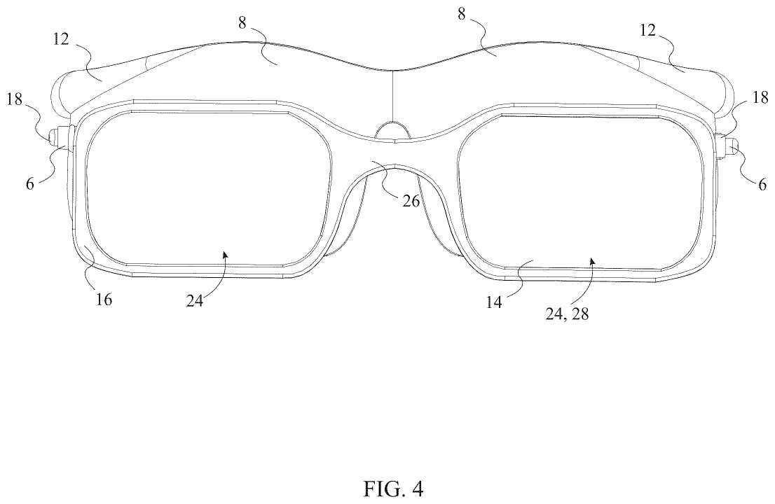

As seen in FIG. 4, the reading frame 16 comprises a pair of lens sockets 24 and a nose bridge 26. The nose bridge 26 is positioned in between the pair of lens sockets 24 connecting the pair of lens sockets 24. Further, each of the pair of reading lenses 14 is positioned within a corresponding socket 28, wherein the corresponding socket 28 is from the pair of lens sockets 24. In other words, the reading eyewear 4 comprises all the parts and looks like a normal cheater glass pair or normal reading glasses, except for the shortened arms 18. Furthermore, each of the pair of shortened arms 18 comprises a first arm end 30 and a second arm end 32, wherein the first arm end 30 is positioned opposite to the second arm end 32 across each of the pair of shortened arms 18. Additionally, as seen in FIG. 3, the reading eyewear 4 further comprises a pair of hinges 34. Preferably, the pair of hinges 34 is hingedly connected between the first arm end 30 of each of the plurality of shortened armes 18 and the reading frame 16. It should be noted that, the pair of reading lenses 14 may comprise any focal power, and the reading frame 16 and the pair of hinges 34 may be made of any material, shape, components, arrangement of components etc. that are known to one of ordinary skill in the art, as long as the intents of the present invention are not altered. According to the present invention, the plurality of fasteners 6 enables securing the reading eyewear 4 to the sides of the safety eyewear 2. As seen in FIG. 7, the plurality of fasteners 6 is positioned adjacent to the second arm end 32.

Continuing with the preferred embodiment, each of the pair of safety lenses 8 further comprises a side shield 36, wherein the side shield 36 extends seamlessly from each of the pair of safety lenses 8. In other words, the side shields are extensions of the pair of safety lenses 8 that protect a user's eyes from the side directions. As seen in FIG. 1, the side shield 36 extends in the direction of a corresponding temple 38, wherein the corresponding temple 38 is from the pair of temples 10. In the preferred embodiment, each of the plurality of fasteners 6 is integrated between a corresponding side shield 40 and a corresponding shortened arm 42, wherein the corresponding shortened arm 42 is from the pair of shortened arms 18. This is so that the side legs of the reading eyewear 4 are positioned along the temples of the user and it does not provide any hindrance while using the reading eyewear 4.

As seen in FIG. 1 through FIG. 3, the safety eyewear 2 further comprises a pair of safety hinges 44, wherein each of the pair of safety hinges 44 is hingedly connected between the corresponding side shield 40 and the corresponding temple 38. In other words, the pair of safety hinges 44 provides the necessary flexibility for the pair of temples and the safety glasses.

It is an aim of the present invention to enable users to quickly and effortlessly attach and detach the reading eyewear 4 from the safety eyewear 2 when needed. To enable this, the present invention comprises a grommet 46, a safety hole 48, and a reading hole 50. In the preferred embodiment, the safety hole 48 traverses through each of the pair of safety lenses 8, and the grommet 46 is integrated into the safety hole 48. Further, the reading hole 50 traverses through each of the pair of shortened arms 18. Furthermore, each of the plurality of fasteners 6 is threaded through the grommet 46 and the reading hole 50. In other words, a pair of holes traverse through the safety eyewear 2 and the reading eyewear 4 to help the user to integrate the fasteners 6 for connecting the reading eyewear 4 on the safety eyewear 2. Preferably, the grommet 46 is made of a flexible material, such as silicone or rubber. This is so that the grommet 46 provides a cushiony yet snug support for the plurality of fasteners 6 while still allowing them to be removed with ease. In the preferred embodiment, the plurality of fasteners 6 comprises screws, or bolts with rounded edges. However, the plurality of fasteners 6 may comprise of any other fastening mechanisms or techniques that are known to one of ordinary skill in the art, as long as the intents of the present invention are not hindered. Examples of such fasteners include, but are not limited to magnetic fasteners, adhesives, other clasp fasteners that allow axial motion of the legs of the reading glasses, etc. Alternatively, the reading eyewear 4 may be attached to the sides of the safety eyewear 2 through just compressive forces and does not even require the plurality of fasteners 6. In order to attach the reading glasses through compression, the distance between the legs of the pair of the reading glasses may be adjusted by adjusting the hinge screws between the glass frame and the legs of the pair of the reading glasses.

In reference to FIG. 1 through FIG. 7, wherein the safety eyewear 2 is in the reading configuration, the reading eyewear 4 is positioned parallel to the safety eyewear 2, and the reading eyewear 4 is laterally offset from the safety eyewear 2.

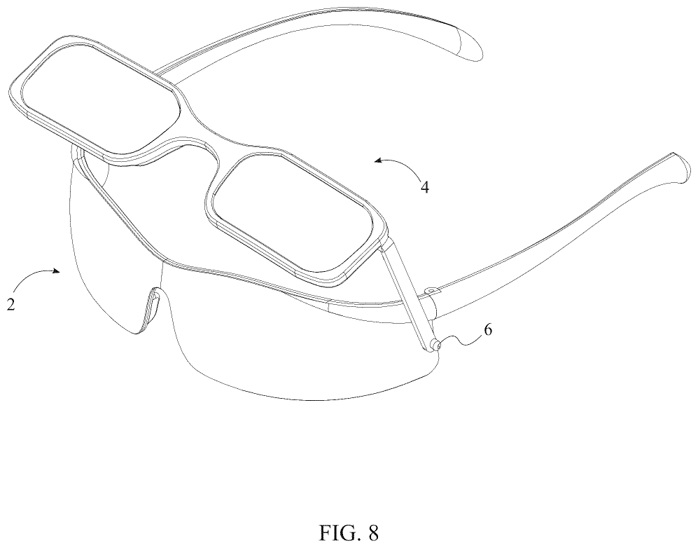

In reference to FIG. 8 through FIG. 10, wherein the safety eyewear 2 is in the non-reading configuration, the reading eyewear 4 is angularly offset from the safety eyewear 2, and the pair of shortened arms 18 is angularly offset from the pair of temples 10.

In the preferred embodiment, the pair of safety lenses 8 of the safety eyewear 2 is transparent, and the pair of reading lenses 14 is smaller than the pair of safety lenses 8. However, the pair of safety lenses 8 may comprise any other tints and the pair of reading lenses 14 may comprise any other size, as long as the intents of the present invention are not altered.

Although the invention has been explained in relation to its preferred embodiment, it is to be understood that many other possible modifications and variations can be made without departing from the spirit and scope of the invention as hereinafter claimed.

What is claimed is:

1. A multifunctional eye wear assembly device comprising:
a safety eyewear;
a reading eyewear;
a plurality of fasteners;
the safety eyewear comprising a pair of safety lenses, a safety frame, and a pair of temples;
the reading eyewear comprising a pair of reading lenses, a reading frame, and a pair of shortened arms;
the pair of temples being terminally mounted onto opposing ends of the safety frame;
the pair of safety lenses being mounted onto the safety frame;
the pair of reading lenses being mounted within the reading frame;
the pair of shortened arms being mounted on to the reading frame;
the reading eyewear being slidably engaged onto the safety eyewear;
the plurality of fasteners being connected between the pair of safety lenses and the shortened arms; and
the reading eyewear being operatively engaged to the safety eyewear through the plurality of fasteners, wherein the safety eyewear slidably switches between a reading mode and a non-reading mode.

2. The multifunctional eye wear assembly device of claim 1, the reading eyewear further comprising:
a pair of hinges;
the reading frame comprising a pair of lens sockets and a nose bridge;
each of the pair of shortened arms comprising a first arm end and a second arm end;
the first arm end being positioned opposite to the second arm end across each of the pair of shortened arms;
the nose bridge being positioned in between the pair of lens sockets connecting the pair of lens sockets;
each of the pair of reading lenses being positioned within a corresponding socket, wherein the corresponding socket is from the pair of lens sockets; and
the pair of hinges being hingedly connected between the first arm end and the reading frame.

3. The multifunctional eye wear assembly device of claim 2, wherein the plurality of fasteners is positioned adjacent to the second arm end.

4. The multifunctional eyewear assembly device of claim 1 comprising:
each of the pair of safety lenses further comprising a side shield;
the side shield extending seamlessly from each of the safety lens; and
the side shield extending in the direction of a corresponding temple, wherein the corresponding temple is from the pair of temples.

5. The multifunctional eyewear assembly device of claim 4, wherein each of the plurality of fasteners is integrated between a side shield and corresponding shortened arm, wherein the corresponding shortened arm is from the pair of shortened arms.

6. The multifunctional eyewear assembly device of claim 4, the safety eyewear further comprising:
a pair of safety hinges;
the pair of safety hinges being terminally mounted onto the safety frame;
each of the pair of safety hinges being hingedly connected between the safety frame and the corresponding temple.

7. The multifunctional eyewear assembly device of claim 1 comprising:
a grommet;
a safety hole;
a reading hole;
the safety hole traversing through each of the pair of safety lenses;
the reading hole traversing through each of the pair of shortened arms;
the grommet being integrated into the safety hole; and
each of the plurality of fasteners being threaded through the grommet and the reading hole.

8. The multifunctional eyewear assembly device of claim 1, wherein the plurality of fasteners are screws.

9. The multifunctional eyewear assembly device of claim 1, comprising:
wherein during the reading mode;
the reading eyewear being positioned parallel to the safety eyewear;
the reading eyewear being laterally offset from the safety eyewear.

10. The multifunctional eyewear assembly device of claim 1, comprising:
wherein during the non-reading mode;
the reading eyewear being angularly offset from the safety eyewear;
the pair of shortened arms being angularly offset from the pair of temples.

11. The multifunctional eyewear assembly device of claim 1, wherein the pair of safety lenses of the safety eyewear is transparent.

12. The multifunctional eyewear assembly device of claim 1, wherein the pair of reading lenses is smaller than the pair of safety lenses.

13. The multifunctional eyewear assembly device of claim 1, wherein a first length of each of the pair of temples is longer than a second length of each of the pair of shortened arms.
